(19) United States
(12) Patent Application Publication
(10) Pub. No.: US 2011/0060412 A1
(43) Pub. Date: Mar. 10, 2011

(54) Tissue Engineered Meniscus Repair Composition

(75) Inventors: Eric J. Semler, Piscataway, NJ (US); Arthur A. Gertzman, Flemington, NJ (US); Yen-Chen Huang, East Brunswick, NJ (US); Judith Yannariello-Brown, Somerset, NJ (US); Katherine Gomes Truncale, Hillsborough, NJ (US)

(73) Assignee: MUSCULOSKELETAL TRANSPLANT FOUNDATION INC., Edison, NJ (US)

(21) Appl. No.: 12/875,829

(22) Filed: Sep. 3, 2010

Related U.S. Application Data
(60) Provisional application No. 61/240,395, filed on Sep. 8, 2009.

(51) Int. Cl. A61K 35/32; A61F 2/08; A61K 9/00; A61P 19/04

(52) U.S. Cl. 623/14.12; 424/400; 424/93.7

(57) ABSTRACT

A meniscus repair composition for application to a meniscus injury to promote growth of new tissue at the meniscus injury site is provided. The composition comprises: from about 10 to about 50 percent by weight of allograft meniscus particles having an average particle size of from about 10 μm to about 500 μm; and a carrier comprising a solid fibrin web matrix. When introduced to a defect site in a meniscus, the composition is non-adhering to the defect site. A method for repairing a meniscus injury comprises administering a meniscus repair composition to the injury site.

### CROSS REFERENCE TO RELATED APPLICATIONS

This application claims priority to U.S. Provisional Application No. 61/240,395, filed Sep. 8, 2009. This Provisional application is incorporated by reference herein, in its entirety and for all purposes.

### TECHNICAL FIELD

The present invention relates generally to the repair and treatment of meniscal injuries. In particular, the present invention relates to a composition and/or an implant comprising the composition wherein the composition comprises allogeneic meniscal tissue and is capable of generating repair tissue at a site of injury to a patient's meniscus once the composition is introduced to the site of injury.

### BACKGROUND

Various publications, including patents, published applications, technical articles and scholarly articles are cited throughout the specification. Each of these cited publications is incorporated by reference herein, in its entirety and for all purposes.

The meniscus plays an important role in load transmission, shock absorption and knee joint stability. Injuries to the meniscus cause pain, disability and damage to the articular cartilage on the femoral and tibial surfaces, leading to development of degenerative changes and osteoarthritis. The meniscus is a dimorphic tissue that consists of two distinctly different tissues, namely the so called “red zone” (vascular) and the so called “white zone” (avascular) tissues.

The red zone, located at the meniscal periphery closest to a vascular blood supply, contains primarily cells that are morphologically fibroblastic. Additionally, the red zone contains a much lesser amount of extra cellular matrix mass than the white zone. Unfortunately, meniscal tears are common in young individuals, usually as a result of sports-related activities, as well as in the older population suffering from degenerative joint diseases. Due to the proximity of the blood supply, lesions, tears and injuries (generally referred to herein as “defects”) in the red zone of the meniscus heal much more rapidly than those occurring in the white zone. Debridement and suturing of the red zone lesions or tears can usually fully restore meniscal function to the red zone, including the restoration of the red zone collagen fibrillar network.

The injuries in the white zone of the meniscus, on the other hand, are currently almost completely untreatable. The white zone itself has no blood supply and is not even located in the proximity of the blood supply. The white zone contains cells that look like chondrocytes typically observed in the articular cartilage, however, the ratio of the extra cellular matrix to cells in the white zone is 10× that of the extra cellular matrix found in the articular cartilage. It is well known that the articular cartilage also does not have any blood supply, that the injuries in the articular cartilage are very difficult to treat, and if they heal at all the ensuing cartilage is an inferior cartilage, called fibrocartilage, rather than normal healthy hyaline cartilage. In this regard the white zone of the meniscus resembles the articular cartilage.

Meniscal defects, particularly those in the white zone, seriously impair the lifestyle of a patient. They can result in altered knee joint function, pain and permanent damage to the adjacent articular cartilage. Due to the avascular nature of the inner white zone region of the meniscus, as described above, a significant number of meniscal lesions or tears do not heal spontaneously. Left untreated, these lesions and tears can propagate into larger defects that exacerbate cartilage damage and the function of the knee.

Early treatments for meniscal injuries typically involve partial or total meniscectomy. This approach frequently results in accelerated cartilage degeneration due to decreased joint contact area and the resultant rise in contact stress. For example, removal of only 15-34% of the meniscus can produce a 350% increase in contact stress. See, e.g., Seedhon B, Hargreaves, D: Transmission of the load in the knee joint with special references to the role of the menisci: II. Experimental results, discussion, and conclusions. Engineering in Med., 8:220 (1979). Therefore, preservation of meniscal tissue and successful lesion repair are the goals of most current treatment methods for meniscal injury.

Currently, a meniscal transplantation is one of the available treatment options for patients whose injury, such as a meniscal tear, is severe and complex. Fresh-frozen allograft menisci have been shown to successfully attach to and heal the recipient periphery in experimental models. Despite these positive results, issues with availability of allograft tissue, tissue rejection, and a lack of long-term data have limited the use of this approach.

Other types of meniscal repair include suturing a torn gap and stapling or employing pins to reapproximate the torn edges. Although typically successful at mending a torn meniscus, these procedures have significant limitations. For example, sutures and pins are typically polymeric materials that comprise polyalphahydroxy acids such as, for example, poly(glycolic acid) and poly(lactic acid). Such polymers are susceptible to degradation, however, to produce their organic acid monomers which may cause bone dissolution.

Yet another method of repairing a meniscus is to glue the torn tissue together with an adhesive such as, for example, the adhesive disclosed in international patent application Publication No. WO 2006/058215 to Kusanagi et al. Adhesives are difficult to work with in that they are unforgiving once applied so the surgeon has little time to manipulate the adhesive at the injury site. Moreover, the adhesives will adhere the torn edges of a meniscal tear together, but the adhesive material per se remains in the site and prevents the regrowth of new, biologically preferred regenerated tissue (e.g., either new meniscal tissue or less desirable fibrocartilage or fibrous “scar” tissue.

Tissue regeneration is recognized as an alternative way to repair a damaged meniscus. For example, international patent application Publication No. WO 2006/064025 to Pastorello et al. discloses providing a polymer matrix comprising a polymer of hyaluronic acid which purportedly induces the repair of damaged meniscal fibrocartilage by providing intercommunicating pores where cells can colonize and proliferate. In addition to the polymer matrix comprising a polymer of hyaluronic acid, WO 2006/064025 relies on a second supporting three-dimensional matrix comprising polymeric fibers to provide the requisite mechanical strength. Although the polymer matrices introduced by a hyaluronic acid does offer a three dimensional matrix space for cells to enter and grow the desired repair tissue, the use of hyaluronic acid, however, is problematical in that it is rapidly metabolized by the patient and will not remain in place long enough for the complex healing to occur. The addition of polymeric fibers will slow the metabolic decay but not prevent the hyaluronic acid matrix from physically breaking down and again precluding the growth of proper repair tissue.

In view of the foregoing, there is a need in the art for a composition for repairing an injured meniscus and regenerating tissue at the damaged site that does not suffer from the above-mentioned drawbacks.

### SUMMARY

The present invention provides compositions for repairing an injured meniscus and regenerating tissue at the damaged site, and methods of repairing an injured meniscus by regenerating tissue by employing such compositions as disclosed below in multiple embodiments.

In some aspects, the invention provides meniscus repair compositions comprising from about 10 to about 50 percent by weight of allograft meniscus particles having an average particle size of from about 10 μm to about 500 μm and a carrier comprising a solid fibrin web, and the compositions are non-adherant to an injury. The compositions may comprise a growth factor such as an allogenic or autologous growth factor, and the growth factor may comprise one or more of TGF-β, VEGF, BMP-2, IGF-1, Nell-1, or TP 508. The allograft meniscus particles may comprise substantially all red zone meniscus particles or substantially all white zone meniscus particles. The solid fibrin web is preferably an autologous solid fibrin web.

The composition may comprise cells, cell extracts, factors expressed by cells, or various agents. For example, the compositions may comprise one or more of a chondrocyte, white blood cell, bone marrow cell, mesenchymal stem cell, pluripotent cell, osteoblast, osteoclast, fibroblast, epithelial cell, or endothelial cell. The solid fibrin web may comprise one or more growth factor additives such as of TGF-beta, IGF-1, PDGF, VEGF, FGF-2, FGF-4, FGF-9, BMP-2, BMP-4, BMP-7, BMP-9, BMP-14, Nell-1, TP 508, osteopontin, or growth hormone, including somatotropin. The composition may comprise one or more of an antiviral agent, amino acid, vitamin, co-factor for protein synthesis, hormone, endocrine tissue or fragment thereof, synthesizer, collagenase, peptidase, oxidase, polymer cell scaffold having parenchymal cells, angiogenic agent, collagen lattice, biocompatible surface active agent, or cartilage. The composition may comprise one or more of a suture, staple, or biological glue.

When administered to a meniscus injury, the compositions facilitate the growth of new meniscus tissue at the meniscus injury. The compositions may facilitate one or more of blood vessel formation, fibrochondrocyte production, cell infiltration, or formation of three-dimensional meniscus tissue at the meniscus injury. The compositions generally facilitate healing of the injury.

In some aspects, the invention provides methods for repairing a knee meniscus injury. Generally, the methods comprise administering a composition such as those described and/or exemplified herein to a site at least proximal or adjacent to the injury. The composition may be administered directly to the injury. Once administered, the composition may be secured at a desired location. Where the composition comprises a suture, the injury may be sutured closed with the suture, with the effect that the composition is secured in place by nature of the suturing. Once administered to a meniscus injury, the compositions facilitate the growth of new meniscus tissue at the meniscus injury. The compositions may facilitate one or more of blood vessel formation, fibrochondrocyte production, cell infiltration, or formation of three-dimensional meniscus tissue at the meniscus injury. The methods generally facilitate healing of the injury.

### BRIEF DESCRIPTION OF SEVERAL VIEWS OF THE DRAWINGS

The invention is best understood from the following detailed description when read in connection with the accompanying figures. It is emphasized that, according to common practice, the various features of the figures are not to scale. On the contrary, the dimensions of the various features are arbitrarily expanded or reduced for clarity. Included are the following figures:

FIG. 1 is a photograph of a composition according to the present invention, and shows human meniscal particles combined with PRFM (human blood).

FIG. 2 shows an SEM micrograph of a composition according to the present invention. Shown are meniscal particles/PRFM with hematoxylin and eosin stain; 200 μm scale.

FIG. 3 shows an SEM micrograph of a composition according to the present invention. Shown are meniscal particles with mesenchymal stem cells, treated 12 days with TGF-beta 1 and visualized with hematoxylin and eosin stain; 100 μm scale.

### DETAILED DESCRIPTION

The present invention generally relates to compositions and methods for repairing an injured meniscus and regenerating tissue at the damaged site. In particular, a composition and method increase the rate of meniscus repair and induce the formation of more normal (i.e., endogenous-type) meniscal tissue than has been commonly observed heretofore. A meniscal repair composition can enhance or otherwise facilitate the body's natural tissue repair processes to repair any injury to a meniscus. The compositions are suitable for repairing any injury, including trauma, mechanical injury, surgical incision, surgical resection, tissue wear, tissue degeneration, or any other injury to the meniscus, from whatever source of the injury.

A meniscal repair composition can induce meniscus repair of avascular tears and fill the injury with meniscus-like tissue. Moreover, a composition and method are useful for repairing and regenerating meniscal tissue which has been removed by partial or complete meniscectomy. A composition and method can enhance blood vessel formation, produce fibrochondrocytes, induce cellular infiltration into the composition, induce cellular proliferation, and produce cellular and spatial organization to form a three-dimensional meniscus tissue.

As used herein, the singular forms “a,” “an,” and “the” include plural referents unless expressly stated otherwise.

The term “implant” is used to refer to tissue, compositions or cells (xenogeneic, autologous or allogeneic) which may be introduced into the body of a patient to replace or supplement the structure or function of the endogenous tissue.

The terms “autologous” and “autograft” refer to tissue or cells which originate with or are derived from the recipient, whereas the terms “allogeneic” and “allograft” refer to cells and tissue which originate with or are derived from a donor of the same species as the recipient. The terms “xenogeneic” and “xenograft” refer to cells or tissue which originate with or are derived from a species other than that of the recipient.

The term “exposing” refers to soaking the tissue in a fluid comprising the treatment agent for a period of time sufficient to treat the tissue. The soaking may be performed by, but is not limited to, incubation, swirling, immersion, mixing, or vortexing.

The term “tissue” is used in the general sense herein to mean any transplantable or implantable tissue, the survivability of which is improved by the methods described herein upon implantation. In particular, the overall durability and longevity of the implant are improved, and host-immune system mediated responses are substantially eliminated. The tissue includes but is not limited to meniscus; ligaments; basal membrane; dermis; tendons; pericardia, cartilage tissue; tubular tissue such as, by way of example and not limitation, arterial tissue and vein tissue; heart valve tissue; demineralized bone tissue; tissues used to construct heart valves such as, by way of example and not limitation, dura mater and pericardium tissue; transparent tissue such as, by way of example and not limitation, cornea and lens tissue; membrane-like tissue such as, by way of example and not limitation, porcine pericardium; bovine pericardium; porcine intestine tissue and lung tissue, more specifically porcine sub-mucosa tissue; bladder tissue; human tissue that is generated and discarded during human childbirth, e.g., human placenta and umbilical cord tissue generated during child birth; amniotic membrane tissue; and the like.

Compositions

One embodiment provides a meniscus repair composition for application to a meniscus defect site to promote growth of new tissue at the meniscus defect site, the composition comprises: a) from about 10 to about 50 percent by weight of allograft meniscus particles having an average particle size of from about 10 μm to about 500 μm; b) a carrier comprising a solid fibrin web, wherein the composition, when introduced to a defect site in a meniscus, will not flow away from the defect site, and wherein the composition is non-adhering to the defect site.

The composition comprises allograft meniscus particles. The allograft meniscus particles function in several ways. For example, the allograft meniscus particles function to provide a matrix (i.e., a physical three-dimensional environment sufficient to act as a scaffold for infiltrating cells to support tissue growth). New meniscus tissue may grow or at least collagen fibrous tissue can fill the space using the meniscal tissue as a matrix upon which the new meniscal tissue will grow. This, in effect, allows for the regeneration of a functional tissue filling a gap or tear in a patient's meniscus. The allograft meniscus particles also function to provide the biochemical cues to initiate a healing response from cells that have either infiltrated the matrix from surrounding host tissue and bleeding bone or from cells that have been added initially to the composition.

The allograft meniscus particles of the composition preferably have an average particle size of from about 10 μm to about 500 μm, more preferably from about 10 μm to about 250 μm, and most preferably from about 10 μm to about 100 μm. In one embodiment, the allograft meniscus particles have a size (e.g., at least one dimension) within a range of from about 10 microns to about 210 microns (i.e., from about 0.01 mm to about 0.21 mm). Alternatively, the allograft meniscus particles may have a size (i.e., the aforesaid at least one dimension) that is within a range of from about 10 microns to about 120 micron (i.e., from about 0.01 mm to about 0.12 mm). The at least one dimension of the allograft meniscus particles may alternatively be less than or equal to 212 microns; within a range of from about 5 microns to about 212 microns; within a range of from about 6 microns to about 10 microns; less than or equal to 5 microns; less than or equal to 10 microns; or less than or equal to 100 microns. In another embodiment, the at least one dimension of most of the particles is less than 100 microns. In yet another embodiment, the at least one dimension of the allograft meniscus particles has a mean and/or median value in the range of between 10 microns and 200 microns. The small size of the allograft meniscus particles can facilitate the increased exposure of, or release of, various growth factors due to the increased aggregate surface area of the particulate allograft meniscus used, and can increase the capacity of the surrounding and infiltrating cells to attach to the allograft meniscus particles.

The allograft meniscus particles can be a mixture of red and white zone allograft meniscus, substantially all red zone allograft meniscus particles, or substantially all white zone allograft meniscus particles. Separation of the allograft meniscus particles into substantially red zone particles or substantially white zone particles yields compositions that benefit from the inherent endogenous chemical composition of each respective anatomical zone. For example, the biochemical composition of a human meniscus comprises a mixture of endogenous growth factors such as, for example, transforming growth factor beta (TGF-β), vascular endothelial growth factor (VEGF), bone morphogenic protein-2 (BMP-2), insulin-like growth factor 1 (IGF-1), thrombin peptide 508 (TP 508), and nel-like molecule 1 (Nell-1). Vascular endothelial growth factor (VEGF), however, is more prevalent in the vascular-containing red zone of the meniscus. Thus, a composition whose allograft meniscal particles comprise substantially all red zone allograft particles is particularly useful in repairing defects in the red zone because they can deliver higher doses of growth factors endogenous to the red zone.

The allograft meniscus particles are preferably prepared by a process that cleans, sterilizes, lyophilizes, and grinds the lyophilized meniscus tissue. In one exemplary embodiment, the allograft meniscus particles are prepared by a process comprising the steps of: disinfecting an allograft meniscus; cutting the allograft meniscus into multiple pieces; lyophilizing the allograft meniscus pieces; grinding the pieces at a temperature of below about 4° C. to achieve ground allograft meniscus particles having the average particle size of from about 10 μm to about 500 μm; and separating unwanted particles by sieving the ground allograft meniscus particles through a sieve having an appropriately sized mesh.

Disinfecting an allograft meniscus may comprise exposing the allograft meniscus to multiple solutions such as, for example, a solution of an oxidizing agent such as, for example, hydrogen peroxide, H₂O₂, an alcohol solution, and optionally a solution of a non-ionic surfactant. In addition to such solutions, it is preferred to also employ frequent intermittent purified water washes. Exposing the allograft meniscus to such solutions is preferably carried out under suitable agitation at a temperature of from below about 34° C.

The oxidizing agent is provided in an aqueous solution and, thus, contains water. The oxidizing agent portion of the solution can be from about 0.5 to about 30 percent by weight of the solution, preferably from about 1 to about 10 percent by weight of the solution and, more preferably, from about 3 to about 5 percent by weight of the solution. Suitable oxidizing agents include, but are not limited to, hydrogen peroxide, periodic acid, peracetic acid, sodium iodate, sodium hypochlorite, and mixtures thereof. Hydrogen peroxide is the preferred oxidizing agent.

The alcohol solution is an aqueous solution and, thus, contains water. The alcohol portion of the solution can be from about 10 to about 90 percent by weight of the solution, preferably from about 20 to about 80 percent by weight of the solution and, more preferably, from about 30 to about 70 percent by weight of the solution. Suitable alcohols include, but are not limited to, ethanol, propanol, iso-propanol, hexanol, and mixtures thereof. A mixture of ethanol and iso-propanol is preferred.

When present, the non-ionic surfactant is provided in an aqueous solution and, thus, contains water. The non-ionic surfactant portion of the solution can be from about 0.01 to about 10 percent by weight of the solution, preferably from about 0.01 to about 3 percent by weight of the solution and, more preferably, from about 0.10 to about 1 percent by weight of the solution. Suitable non-ionic surfactants include, but are not limited to, Triton® X-100 (Union Carbide Corp., NY), Tween® 80 (ICI Americas, Inc., DE), N, N-dimethyldodecylamino-N-oxide, octylglucoside, polyoxyethylene (PEG) alcohols, polyoxyethylene-p-t-octylphenol, polyoxyethylene nonylphenol, polyoxyethylene sorbitol esters, polyoxy-propylene-polyoxyethylene esters, p-iso-octylpolyoxy-ethylene-phen-ol formaldehyde polymer, and mixtures thereof. Triton® X-100 is the preferred non-ionic surfactant.

In some embodiments, the disinfecting step further comprises exposing the allograft meniscus or meniscus pieces to an antibiotic solution. Preferred antibiotics include, for example, gentamicin, erythromycin, bacitracin, neomycin, penicillin, polymyxin B, tetracycline, viomycin, chloromycetin and streptomycin, cefazolin, ampicillin, azactam, tobramycin, triclosan, clindamycin, and mixtures thereof.

In some embodiments, the disinfecting step further comprises exposing the allograft meniscus or meniscus pieces to a buffered saline solution to ensure removal of the above-identified process solutions. Preferably, the saline solution is buffered at a pH of about 6.5 to about 7.8 and, more preferably from about 7.2 to about 7.4.

Preferably, the disinfecting step functions to remove antigenic elements, residual cellular debris, and lipids from the allograft meniscus or allograft meniscus pieces. Exposure time to each of the above-identified solutions, if employed, can be anywhere from 1 minute to 24 hours, preferably, from 1 hour to 8 hours, and more preferably from 3 to 5 hours. The order of the steps of the disinfecting process are not critical to the invention; however, exemplary processes are disclosed in U.S. patent application Publication No. 2004/0037735, which is incorporated herein by reference.

The process includes the step of cutting the allograft meniscus into multiple pieces. This step can be performed before or after the above-described disinfecting step. Any suitable sterile cutting means known in the art such as, for example, a scissor or scalpel, can be employed to cut the allograft into multiple pieces. Preferably the allograft meniscus is cut into pieces of from about 85 to about 300 μm of irregularly-shaped polygonal particles.

The process includes the step of lyophilizing the allograft meniscus pieces. Those skilled in the art will appreciate that lyophilization is a freeze-drying process in which water is sublimed from the composition after it is frozen. The particular advantage associated with the lyophilization process is that biological materials can be dried without elevated temperatures, thereby eliminating the adverse thermal effects. An exemplary lyophilization process includes an initial shelf temperature of from about −20° C. to about −55° C., and preferably about −40° C. for about 4 hours, with the temperature raised to +35° C. for about 28 hours, with the last 29 hours being under a vacuum of about 350 mTorr.

The process includes the step of grinding the pieces of allograft meniscus at a temperature of from less than about 4° C. to achieve ground allograft meniscus particles having the average particle size of from about 10 μm to about 500 μm, more preferably from about 10 μm to about 250 μm, and most preferably from about 10 μm to about 100 μm. The allograft meniscus particles are preferably cryogenically ground (i.e., below −185° C.) to achieve such desired particle size. In some embodiments, the ground lyophilized meniscus tissue is sieved through an appropriately sized mesh screen to achieve the desired average particle size. Employment of a sieve is an optional component. The average particle size of the allograft meniscus particles is determined by methods well known to the skilled artisan such as, for example, with an ImagePro Plus® (Media Cybernetics Inc., MD) software with optional microscope.

Such processes described above are preferably performed in a manner which ensures the efficacy of the processed tissue for introduction into a human patient. Accordingly, the processes are preferably performed in a sterile environment such as, for example, a Class 10 clean room. More preferably, the processed tissue is, at some point prior to introduction into a human patient, further sterilized by exposure to radiation such as, for example, gamma or electron-beam radiation at a dose of from about 3 to about 30 kiloGreys.

The compositions include a carrier comprising a platelet rich fibrin matrix gel (also referred to herein as “PRFM” or “solid fibrin web”). The solid fibrin web is preferably obtained by operation of the Cascade® PRFM system, which is marketed by the Musculoskeletal Transplant Foundation, Edison, N.J. In such system, a solid fibrin web is obtained by drawing blood from a patient into a primary container and separating plasma from the blood in the primary container via centrifugation. Plasma from the primary container is transferred to a secondary container containing a coagulation activator, for example, a calcium compound (for example, calcium chloride), using a transfer device comprising a cannula having a first end and a second end in order to contact the plasma with the coagulation activator. The plasma and coagulation activator are then concurrently coagulated and centrifuged in the secondary container in order to form the solid-fibrin web, which is suitable for implantation into the patient. The methods, apparatus and materials for making the solid fibrin web of the present invention are disclosed in U.S. Pat. Nos. 6,979,307 and 6,368,298, as well as U.S. patent application Publication No. 2006/0074394, the disclosures of which are incorporated herein by reference in their entireties.

The solid-fibrin web carrier is advantageous because of its inherent ability to promote the formation of new tissue. In this regard, the repair response of musculoskeletal tissues generally starts with the formation of a blood clot and degranulation of platelets, which releases growth factors and cytokines at the site. This microenvironment results in chemotaxis of inflammatory cells as well as the activation and proliferation of local progenitor cells. In most cases, fibroblastic scar tissue is formed. In some settings, however, such as in a fracture callus, these conditions can also facilitate the formation of new tissue. The following endogenous growth factors can be found in the environment of a blood clot: transforming growth factor beta (TGF-β); platelet-derived growth factor (PDGF); insulin-like growth factor (IGF); vascular endothelial growth factors (VEGF); epidermal growth factor (EGF); and fibroblast growth factor-2 (FGF-2). Autologous solid-fibrin web is preferred because it contains a biologically active mixture of growth factors without the potential for an immune response. Additional (i.e., non-inherent) growth factors may also be added to the PRP as described in more detail below.

The solid fibrin web comprises within its gel-like matrix the ground allograft meniscus particles. The primary role of the solid fibrin web is to serve as a delivery vehicle for the allograft meniscus particles. The ground allograft meniscus particles can be added to the plasma prior to mixing it with the coagulation activator or after mixing the plasma with the coagulation activator. Homogeneous mixing can be obtained by any suitable means known in the art. In preferred embodiments of the present invention, the secondary container includes, in addition to the coagulator, the ground allograft meniscus particles such that, when mixed with the plasma and centrifuged in the secondary container, the solid web includes the allograft meniscus particles.

Preferably, the solid fibrin web comprises from about 10 to about 50 percent by, weight of the ground allograft meniscus particles. More preferably, the solid fibrin web comprises from about 5 to about 35 percent by weight of the ground allograft meniscus particles. Most preferably, the solid fibrin web comprises from about 10 to about 25 percent by weight of the ground allograft meniscus particles.

Additives that are beneficial to tissue growth may be added to the compositions at any stage of the mixing process. Such additives include living cells and cell elements such as chondrocytes, white blood cells, bone marrow cells, mesenchymal stem cells, pluripotent cells, osteoblasts, osteoclasts, and fibroblasts, epithelial cells, and endothelial cells. These cells or cell elements or combinations of the same are typically present at a concentration of 10⁵ to 10⁸ per cc of carrier and are added into the composition at the time of surgery. In a preferred embodiment, the compositions comprise autologous bone marrow cells aspirated from the patient during a surgical procedure to repair a defective meniscus with the composition.

Growth factor additives can also be added to the compositions either at the time of packaging the secondary container or at surgery, depending on the stability of the growth factor. Such growth factors include, but are not limited to transforming growth factor beta (TGF-β), insulin growth factor (IGF-1); platelet derived growth factor (PDGF), vascular endothelial growth factor (VEGF), fibroblast growth factor (FGF) (numbers 1 to 23 and, in particular, numbers 2, 4 and 9), bone morphogenic factors (BPM 2, 4, 7, 9 and 14), Nell-1, TP 508, osteopontin, and growth hormones such as somatotropin cellular attractants.

Any number of medically useful substances can also be added to the compositions such as, for example, antiviral agents (such as those effective against HIV and hepatitis), amino acids, polypeptides, vitamins, co-factors for protein synthesis, hormones, endocrine tissue or fragments thereof, synthesizers, enzymes (such as collagenase, peptidases, oxidases), polymer cell scaffolds with parenchymal cells, angiogenic drugs and polymeric carriers containing such drugs, collagen lattices, biocompatible surface active agents, antigenic agents, cytoskeletal agents, cartilage, and cartilage fragments.

The consistency of the compositions is that of a solid but flowable gel, like, for example, toothpaste. Thus, once prepared in the operating room, the surgeon, using a cutting instrument, can shape the composition to exactly fit a meniscal defect. Once shaped, the compositions are then secured to the defect site by a securing mechanism such as, for example, sutures, staples or a biological glue. Thus, the compositions can comprise one or more sutures, staples, or biological glues. Suitable biological glue can be found commercially, such as for example, TISSEEL® (Baxter Int'l, Inc., DE) or TISSUCOL (fibrin based adhesive; Immuno AG, Austria), Adhesive Protein (Sigma Chemical, USA), and Dow Corning Medical Adhesive B (Dow Corning, USA). The composition remains there until the tear or lesion closes and heals, typically within several weeks or months. Since the tear or lesion gap is filled, there is no friction between the two sides of the tear or lesion, there is no further deterioration or enlargement of the tear, nor is there an accompanying deterioration of the adjacent articular cartilage.

The surprising and unexpected effect of the compositions and methods is that actual meniscus or meniscus-like new tissue will grow in and on the allograft meniscus particles. Thus, the compositions, once administered, enhance or otherwise facilitate growth of new meniscus tissue at the site of injury. The compositions facilitate the body's natural healing processes. The compositions may facilitate blood vessel formation, fibrochondrocyte production, infiltration of cells into the injury, and other repair processes. The patient's adjacent meniscus and/or microvasculature will provide mesenchymal stem cells to the site using the implanted meniscal particles as a matrix to enter the damaged (i.e., defective) space and engineer new meniscal tissue. The mesenchymal stem cells can differentiate their environment and proliferate into meniscal cells filling the gap with regenerated meniscal tissue and providing relief from the preoperative pain experienced by the patient.

The compositions are not adhesive compositions and, thus, are non-adhering to surfaces such as, for example, the surfaces of a meniscal defect site, after the composition is cured. Thus, manipulation of the composition may be done without it sticking to the gloves of the surgeon.

In a preferred embodiment, the composition comprises a solid fibrin web matrix comprising substantially red zone meniscus particles. Such composition is preferably employed to repair meniscal defects in the red zone of a patient's meniscus.

Preformed Meniscal Implants

Another embodiment provides a rigid or semi-rigid preformed meniscal implant comprising an above-described composition. The shape of the preformed implant can be, for example, a wedge, a crescent shape, a disc, or a block. Such shapes can be further trimmed by a surgeon to fit a prepared torn space and sutured or glued in place. To make such embodiments, any of the compositions as described above is mixed and placed into a mold immediately after mixing in the secondary container.

Additional objects, advantages, and novel features of these inventions will become apparent to those skilled in the art upon examination of the following examples. The examples are included to more clearly demonstrate the overall nature of the inventions and, thus, are illustrative and not restrictive of the inventions.

### EXAMPLES

### Meniscus Processing (Generally)

Both the lateral and medial meniscus are recovered from the left and right knees of a donor by blunt dissection. At this point, the meniscus may be further dissected into a substantially red zone section and a substantially white zone section. Any residual soft tissue is removed and then each meniscus is subjected to a serious of chemical soaks and rinses. In one embodiment, the meniscal tissue is first soaked in an antibiotic solution containing gentamicin, primaxin and amphotericin B for up to 4 hours at 20-40° C. under agitation, followed by multiple rinses in a saline buffer. In another embodiment, the tissue is also subsequently soaked in a detergent (such as Polysorbate 80 or Triton® X-100) or dilute acid (such as HCl, acetic acid, or peracetic acid) or base (such as NaOH) to further clean the tissue. Single or multiple soaks may be performed for up to 1 hour at 20-40° C. under agitation. More specifically, the tissue can be soaked in 0.1% Triton® X-100 for 15-30 minutes on a reciprocating or orbital shaker at a temperature of approximately 37° C. Following this soak, the meniscal tissue is rinsed multiple times with a saline buffer to remove Triton residuals prior to further processing.

Subsequently, each meniscus is cut into pieces that are approximately no more than 5 mm by 5 mm with a thickness of no more than 5 mm. Cutting of the meniscus can be performed using a scalpel or with a semi-automated or an automated chopping device. The meniscus strips are then lyophilized to a residual moisture level of less than 6% wt/wt. After dehydration, meniscus strips are then subjected to a pulverization process under liquid nitrogen using a freezer milling device (Spex CertiPrep, Metuchen, N.J.). In one embodiment, the milled, pieces are sieved to obtain a particle size of less than 212 microns. In another embodiment, the milled pieces are sieved to a particle size of less than 850 microns. Intone embodiment, these particles are then stored in this dehydrated state until reconstitution. In another embodiment, these meniscal particles are then further cleaned by soaking in a detergent, dilute acid or base, or disinfecting agent such as hydrogen peroxide or ethanol under agitation. After additional chemical soaks, saline rinses are performed to remove residuals and then the particles would be again lyophilized to a residual moisture level of less than 6% wt/wt.

For reconstitution, meniscal particles can be mixed with saline or combined with a carrier. In one example, meniscal particles are reconstituted in sodium hyaluronate to a concentration of 20-45% wt/wt. Reconstitution of the allograft meniscus particles prior to their use in the compositions of the present invention, however, is optional.

### Example 1

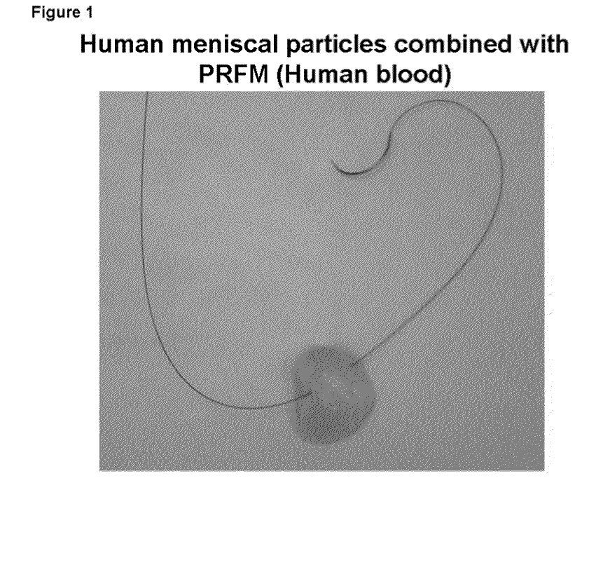

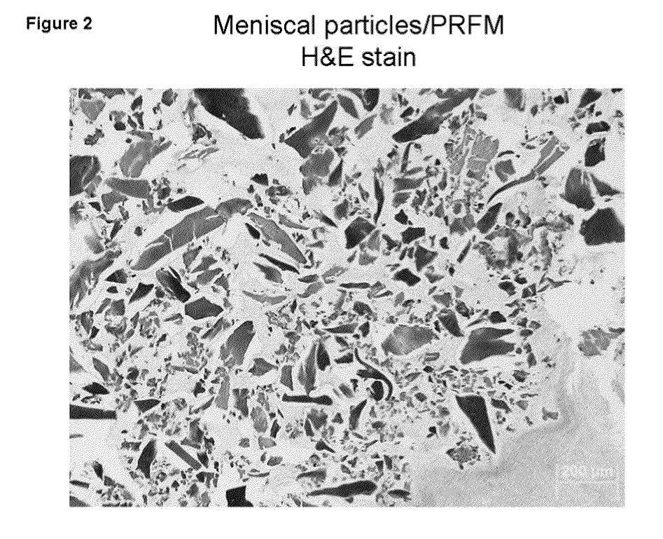

Freezer milled meniscus particles were mixed with autologous platelet rich plasma isolated from a patient using the Cascades® Platelet Rich Fibrin Matrix (PRFM) kit. 9 cc of the patient's blood was drawn into a tube containing an inert, polyester separator gel and tri-sodium citrate anticoagulant. The tube was gently inverted seven times, and centrifuged for six minutes at 1100 g, after which the tube was again gently inverted seven times. The tube was then held vertically, connected to another tube containing calcium chloride through a transfer device. The platelet rich plasma in the former tube was then transferred to and combined with calcium chloride in the latter tube. Meniscal particles ranging from 2% to 50% (w/v) were added to the mixture in the tube, and centrifuged for fifteen minutes at 1450 rpm. The resultant PRFM/meniscal particle matrix (FIG. 1) demonstrated a solid and gel-like appearance that was penetratable by a suture. The histological results confirmed that the meniscal particles were held together by the PRFM (FIG. 2).

The above-described composition can either be injected arthroscopically to the meniscal injured site, or passed through an arthroscopic device and be sutured to existing torn or injured meniscus for repair.

### Example 2

Separated platelet rich plasma in the first tube as described in Example 1 was transferred to a glass bottle container and combined with calcium chloride. Meniscal particles ranging from 1% to 50% (w/v) were added to the mixture in the bottle and centrifuged for twenty five minutes at 3600-4500 rpm. The resultant PRFM/meniscal particle matrix demonstrated a membrane-like structure with meniscal particles interspersed homogeneously throughout the matrix. This matrix can be sutured to repair torn meniscus.

### Example 3

From about 2.5×10⁵ to 2.5×10⁷ bone marrow derived mesenchymal stem cells which have been grown or expanded from a human donor ranging from 3 months to 45 years of age can be inserted by syringe into the solid fibrin web matrix before, during or after deposit of the PRFM/meniscal particle matrix into the defect area. This composite material can be injected into the injured site arthroscopically and fit into the injured site where it is held in place by its own viscosity, or covered and sealed with a biological glue. The matrix can also be combined with growth factors including transforming growth factor-β1 (TGF-β1), fibroblast growth factor-2 (FGF-2), insulin growth factor-1 (IGF-1), and platelet derived growth factor-bb (PDGF-bb), that have been implicated in meniscal repair.

### Example 4

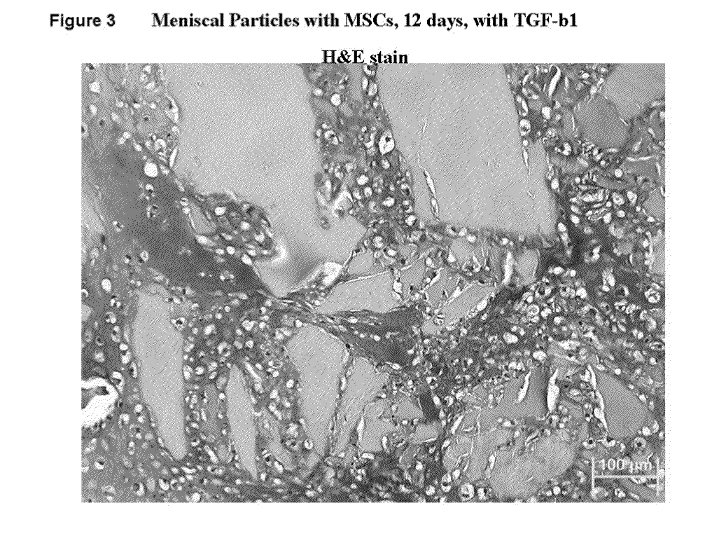

The composition of Example 3 was placed in a 2% agarose gel mold in the presence of chondrogenic growth factors, such as transforming growth factor-β1 (TGF-β1), to assess the in vitro biocompatibility of meniscal particles. The histological data from hemotoxylin and eosin stain demonstrated that the meniscal particles supported cell adhesion and proliferation, and further differentiation of chondrocyte-like cells, which were embedded in lacunae. New cartilage-like matrix formation was also evident by the intense eosin stain, and exhibited seamless integration with meniscal particles (FIG. 3).

### Example 5

Human allograft meniscus is harvested from human donors by blunt dissection. The tissue is typically decellularized through a series of chemical treatment steps. For example, the tissue can be placed in a 1N NaCl solution for 24 hours, followed by 24 hours of a 0.1%-3% Triton X-100 solution soak. The tissue is then typically disinfected by exposure to a 0.5% to 5% peracetic acid solution for a period of from about 2 to 24 hours, followed by several rinses with DI water. The extent of decellularization can be confirmed by histology and residual DNA assessment. The decellularized meniscus is then ready to be combined with PRFM for use in accordance with the present invention.

The principles, preferred embodiments and modes of operation of the present inventions have been described in the foregoing specification. The inventions should not be construed as limited, however, to the particular embodiments which have been described above. Instead, the embodiments described here should be regarded as illustrative rather than restrictive. Variations and changes may be made by those skilled in the art without departing from the scope as defined by the claims that follow. It is expressly intended, for example, that all ranges broadly recited in this document include within their scope all narrower ranges which fall within the broader range.

1. A meniscus repair composition, comprising:
(a) from about 10 percent to about 50 percent by weight of allograft meniscus particles having an average particle size of from about 10 μm to about 500 μm; and,
(b) a carrier comprising a solid fibrin web;
wherein the composition, when administered to a knee meniscus injury, does not adhere to the injury.

2. The composition of claim 1, further comprising a growth factor.

3. The composition of claim 2, wherein the growth factor is an allogenic growth factor.

4. The composition of claim 2, wherein the growth factor is an autologous growth factor.

5. The composition of claim 2, wherein the growth factor comprises one or more of TGF-β, VEGF, BMP-2, IGF-1, Nell-1, or TP 508.

6. The composition of claim 1, wherein the allograft meniscus particles comprise substantially all red zone meniscus particles.

7. The composition of claim 1, wherein the allograft meniscus particles comprise substantially all white zone meniscus particles.

8. The composition of claim 1, wherein the solid fibrin web is autologous solid fibrin web.

9. The composition of claim 1, further comprising one or more of a chondrocyte, white blood cell, bone marrow cell, mesenchymal stem cell, pluripotent cell, osteoblast, osteoclast, fibroblast, epithelial cell, or endothelial cell.

10. The composition of claim 1, wherein the solid fibrin web comprises a growth factor additive.

11. The composition of claim 10, wherein the growth factor additive comprises one or more of TGF-beta, IGF-1, PDGF, VEGF, FGF-2, FGF-4, FGF-9, BMP-2, BMP-4, BMP-7, BMP-9, BMP-14, Nell-1, TP 508, osteopontin, or somatotropin.

12. The composition of claim 1, further comprising one or more of an antiviral agent, amino acid, vitamin, co-factor for protein synthesis, hormone, endocrine tissue or fragment thereof, synthesizer, collagenase, peptidase, oxidase, polymer cell scaffold having parenchymal cells, angiogenic agent, collagen lattice, biocompatible surface active agent, or cartilage.

13. The composition of claim 1, further comprising a suture, staple, or biological glue.

14. The composition of claim 1, wherein the composition facilitates the growth of new meniscus tissue at the meniscus injury when the composition is administered to the meniscus injury.

15. The composition of claim 1, wherein the composition facilitates blood vessel formation, fibrochondrocyte production, cell infiltration, or formation of three-dimensional meniscus tissue at the meniscus injury when the composition is administered to the meniscus injury.

16. A method for repairing a knee meniscus injury, comprising administering a composition according to claim 1 proximal to the injury.

17. The method of claim 16, comprising administering the composition to the injury.

18. The method of claim 16, further comprising securing the composition to the knee meniscus.

19. The method of claim 16, wherein the composition comprises a suture, and further comprising suturing the injury with the suture.

20. The method of claim 16, wherein the composition facilitates the growth of new meniscus tissue at the meniscus injury after administering the composition to the meniscus injury.
